(19) United States
(12) Patent Application Publication
(10) Pub. No.: US 2019/0125983 A1
(43) Pub. Date: May 2, 2019

(54) PREEMPTIVE PAIN AVOIDANCE AND/OR DIAGNOSTIC APPARATUS AND METHODS OF OPERATING SAME

(71) Applicant: NEWTONOID TECHNOLOGIES, L.L.C., LIBERTY, MO (US)

(72) Inventors: FIELDING B. STATON, LIBERTY, MO (US); DAVID STRUMPF, COLUMBIA, MO (US)

(21) Appl. No.: 16/172,240

(22) Filed: Oct. 26, 2018

Related U.S. Application Data
(60) Provisional application No. 62/577,226, filed on Oct. 26, 2017.

(51) Int. Cl. A61M 5/42; A61H 23/00

(52) U.S. Cl. CPC A61M 5/422; A61C 3/00; A61H 23/00

(57) ABSTRACT

A method for distracting an individual from a treatment event comprises providing a distraction tool; positioning the distraction tool at a contact location on an individual, the contact location being substantially adjacent a location of a treatment event; activating an oscillation device to induce vibrations in the distraction tool; and subsequently initiating the treatment event at the treatment event location. The distraction tool comprises a stimulation apparatus comprising a housing and a plurality of projections extending from the housing. The oscillation device is in communication with the distraction tool to induce vibrations specifically in the stimulation apparatus. Activating the oscillation device to induce vibrations causes the plurality of projections to oscillate against the individual at the contact location, thereby shifting attention from the treatment event to the vibrations, wherein the shift in attention causes the individual to discount pain caused by the treatment event.

### CROSS-REFERENCE TO RELATED APPLICATIONS

This application claims priority to U.S. Provisional Application No. 62/577,226, filed Oct. 26, 2017, the entirety of which is incorporated herein by reference.

### BACKGROUND

Humans involuntarily react to situations that suggest that the body is in danger. For example, when an injection is given (e.g., vaccinations), a person may experience slight pain at the injection site when the needle penetrates the skin. The pain is in response to the body's belief that it is experiencing an unwanted circumstance. If the body can be prepared in advance for an upcoming event, then the event may be less traumatic for the person (e.g., reduced pain).

### SUMMARY

The following presents a simplified summary of the invention to provide a basic understanding of some aspects thereof. This summary is not an extensive overview of the invention. It is not intended to identify critical elements of the invention or to delineate the scope of the invention. Its sole purpose is to present some concepts of the invention in a simplified form as a prelude to the more detailed description that is presented elsewhere herein.

In one embodiment, a method for distracting an individual from a treatment event comprises providing a distraction tool; positioning the distraction tool at a contact location on an individual, the contact location being substantially adjacent a location of a treatment event; activating an oscillation device to induce vibrations in the distraction tool; and subsequently initiating the treatment event at the treatment event location. The distraction tool comprises a stimulation apparatus comprising a housing and a plurality of projections extending from the housing. The oscillation device is in communication with the distraction tool to induce vibrations specifically in the stimulation apparatus. Activating the oscillation device to induce vibrations causes the plurality of projections to oscillate against the individual at the contact location, thereby shifting attention from the treatment event to the vibrations, wherein the shift in attention causes the individual to discount pain caused by the treatment event.

In another embodiment, a method for distracting an individual from a treatment event comprises providing a distraction tool; positioning the distraction tool at a contact location on an individual; activating an oscillation device to induce vibrations in the distraction tool; and subsequently initiating the treatment event to the individual. The distraction tool comprises a stimulation apparatus and an oscillation device. The oscillation device is in communication with the distraction tool to induce vibrations specifically in the stimulation apparatus. Activating the oscillation device to induce vibrations causes the distraction tool to vibrate against the individual, thereby shifting attention from the treatment event to the vibrations, wherein the shift in attention causes the individual to discount pain caused by the treatment event.

In still another embodiment, a system for diagnosing and providing a distraction to an individual comprises a distraction tool, and a computing structure. The distraction tool includes a stimulation apparatus comprising a housing and a contact area having a distraction mechanism, and a sensor. The computing structure comprises an input/output device and a processor in data communication with non-transitory computer memory. The computer memory includes a program that, when effectuated by the processor, performs the following steps: (1) receive a first input from the input/output device to determine a first level of distraction; (2) activate the distraction mechanism of the stimulation apparatus based on the first level of distraction; (3) receive a second input from the input/output device to determine a second level of distraction; (4) adjust the distraction mechanism of the stimulation apparatus based on the second level of distraction; (5) receive a sensor input from the sensor; (6) analyze the sensor input based on a predetermined set of conditions; and (7) provide an output at the input/output device, the output being based on the sensor input analysis.

### BRIEF DESCRIPTION OF THE DRAWINGS

FIG. 1 is a top view of a preemptive pain avoidance apparatus according to an embodiment of the invention.

FIG. 2 is a top view of a preemptive pain avoidance and diagnostic apparatus according to another embodiment of the invention.

FIG. 3 is a perspective view of a preemptive pain avoidance apparatus according to yet another embodiment of the invention.

FIG. 4 is a side view of a preemptive pain avoidance and diagnostic apparatus according to an embodiment of the invention.

FIG. 5 is a top view of a preemptive pain avoidance apparatus according to still another embodiment of the invention.

FIG. 6 is a bottom view of the preemptive pain avoidance of FIG. 5.

FIG. 7 is a side view of the preemptive pain avoidance apparatus of FIG. 5.

FIG. 8 is a side view of the preemptive pain avoidance apparatus of FIG. 5 with a plug removed.

FIG. 9 is a side view of the plug removed from the preemptive pain avoidance apparatus of FIG. 8.

FIG. 10 is a block diagram of an analysis platform in communication with a preemptive pain avoidance and/or diagnostic apparatus according to an embodiment of the invention.

### DETAILED DESCRIPTION

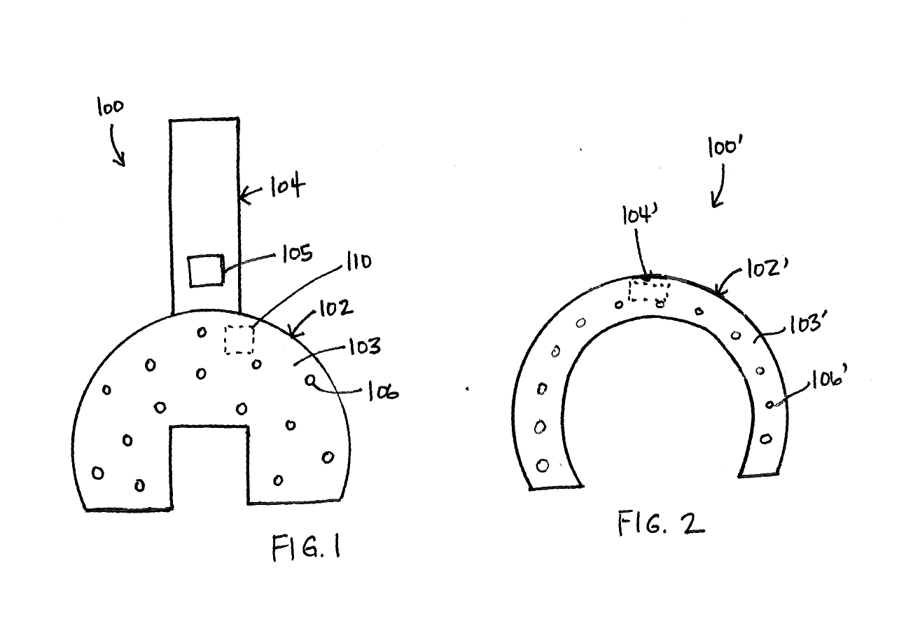

Preemptive pain avoidance and diagnostic apparatus and methods are described herein with reference to FIGS. 1-10. In one embodiment, a preemptive pain avoidance apparatus is a mechanical device designed to contact the surface of the skin of an individual in need of a treatment (e.g., injection) and distract the individual. FIGS. 1-3 illustrate exemplary embodiments of a distraction apparatus 100, 100′, and 100″. The apparatus 100, 100′, and 100″ each include a stimulation apparatus 102, 102′, and 102″, and an oscillation device 104, 104′, and 104″, respectively. The apparatus 100′ and 100″ are substantially similar to apparatus 100 except as shown and described herein, and therefore the reference numbers given to the various components in embodiments 100′ and 100″ correspond to the components in embodiment 100. For example, the housings 103′ and 103″ are substantially similar except as may be shown or described. For ease of review, reference numerals referring only to components of embodiment 100 are utilized below. However, it shall be understood that the components of embodiments 100′ and/or 100″ are also included within the description except as explicitly set forth herein.

The stimulation apparatus 102 includes a housing 103 optionally having a cutout 103a formed therein. In an embodiment, the cutout 103a is configured to allow a needle or other instrument to pass therethrough. The housing 103 may be configured as a plate, although other configurations may be possible and are contemplated within the scope of the invention. In one configuration, such as that shown in FIG. 1, the stimulation apparatus 102 may be horseshoe shaped such that it extends substantially around an area where the treatment event will occur. In another embodiment, as shown in FIG. 3, the housing 103″ may be configured as a sleeve to fit around a user's arm or leg. Or, as shown in FIG. 2, the housing 103′ may be configured as a mouthpiece to fit in a user's mouth. In other embodiments, the stimulation apparatus 102 may have a donut shape (which may be round, square, oval, polygonal, etc.) or any other appropriate shape.

The oscillation device 104 may be incorporated into a handle, such as shown in FIG. 1. In other embodiments, the oscillation device 104 is integral with the stimulation apparatus 102. In any event, the oscillation device 104 includes a motor 105 which may be activated to induce oscillations.

A plurality of projections 106 may extend from the housing 103 which contact and engage with an individual to distract the individual from the treatment event. Each projection 106 provides a separate source of contact between the stimulation apparatus 102 and the individual's skin. In one embodiment, the projections 106 are flexible. When the stimulation apparatus 102 is activated via the oscillation device 104, each of the flexible projections 106 may oscillate separate independently of one another. The projections 106 may additionally be temporarily deformed upon contact with the individual's skin. Because each of the projections 106 flex independently of the others, an individual using stimulation apparatus 102 may experience several distraction points. In other words, because each projection 106 can move and flex on its own, the user may feel different sensations at each point of contact with the user's skin. This may provide a perceived heightened level of distraction for the individual experiencing the treatment event.

In another embodiment, the projections 106 are rigid. Here, fewer projections 106 may be required, and each of the projections 106 oscillate together with the housing 103 to distract the individual experiencing the treatment event. Rigid projections 106 may be preferable where the treatment event is of a greater magnitude. While the force of the oscillations induced by the oscillation device 104 may be somewhat diminished where the projections 106 are flexible, where the projections 106 are rigid, the force of the oscillations is passed directly to the individual. Therefore, because the projections 106 are rigid, they do not deform when in contact with the individual, and act in concert to provide more intense distraction to the individual.

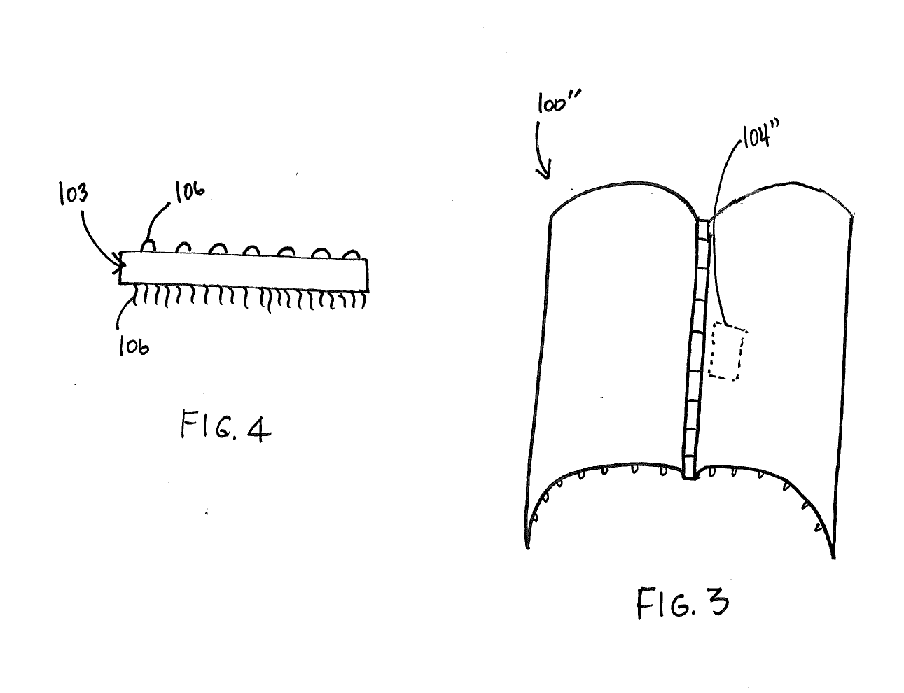

In still another embodiment, the housing 103 may include both rigid and flexible projections 106. For example, rigid projections 106 may extend from a first side of the housing 103, and flexible projections may extend from a second side of the housing 103, as shown in FIG. 4. Accordingly, the same stimulation device 102 may be utilized as a distraction mechanism for many types of treatment events. In a further embodiment, both rigid and flexible projections 106 may be located on a single side of the housing 103.

Regardless of the level of rigidity, when the projections 106 contact the individual (e.g., the skin), the individual's focus turns to the primary source of disruption—in this case, the contact with the stimulation apparatus 102—rather than the treatment event. It may thus be possible to provide a distraction which is sufficient to prevent an individual from feeling pain in situations where the individual would otherwise experience some level of discomfort. It is believed that in certain circumstances, the distraction device 102 can provide enough of a distraction that other, more intense, distraction techniques (e.g., sedatives such as nitrous oxide or types of anesthesia), would not be required. This could be significant for individuals that experience allergies or do not respond well to chemical distraction techniques, or may otherwise choose to undergo a treatment event without any form of distraction.

The stimulation apparatus 102, and particularly the housing 103, may be fabricated from any material, and preferably from a hypoallergenic material, such as surgical steel, nylon (e.g., cellulose acetate propionate), zylonite, etc. In embodiments, it may be preferable for the housing 103 to be constructed of a material that can retain heat or coldness. The housing 103 may be heated or cooled to a particular temperature before placing the housing 103 in contact with an individual. The temperature differential between the individual's body and the housing 103 may provide yet another level of distraction. In embodiments, a heating and/or cooling mechanism 110 may be integral with the housing 103, and may be activated via a switch.

The stimulation apparatus 102 is attached, either permanently or temporarily, to the oscillation device 104. In embodiments, the oscillation device 104 is integral with the stimulation apparatus 102. When activated, the oscillation device 104 causes the stimulation apparatus 102 to vibrate or oscillate. The oscillation device 104 may simply switch between on and off states. Alternately, the oscillation device 104 may have modes (e.g., slow vibration, medium vibration, intense vibration, etc.) which allow a user to select the intensity of the oscillations based on the treatment event (e.g., finger prick, injection, etc.). For example, for a minor finger prick to draw a small amount of blood (e.g., to test blood glucose levels), only low intensity vibrations may be required in order provide a sufficient distraction that the individual does not feel pain when stuck with a needle, as the needle is very small. However, injections utilizing larger needles may require more intense vibrations. Other treatment events (which may or may not include medical devices) may additionally call for some form of distraction. For example, adjustments in the musculoskeletal system (e.g., chiropractic adjustments) may be uncomfortable for some people; accordingly, the distraction apparatus 100 may be provided to distract the individual from such treatment events.

The distraction apparatus 100 may be used simultaneously with an event that an individual may be experiencing. In the example of an injection, the apparatus 100 may be placed at an injection site such that the stimulation apparatus 102 is in contact with the individual's skin near the injection site. While the apparatus 100 is oscillating against the individual's skin, the injection is administered to the individual. The oscillations distract the individual and potentially decrease the pain experienced by the individual. Where the treatment event does not include an injection, the distraction apparatus 100 may be placed at an appropriate location on the individual according to the treatment event.

Many individuals experience anxiety when visiting the dentist. The anxiety is often related to the very uncomfortable nature of how the dentist goes about assessing issues with the patient's teeth, as there is often no easy way in which a patient can precisely point the dentist to the problem. For example, a person experiencing a tooth ache may not know exactly which tooth is causing the pain. The patient may experience pain in a focused area, or the patient may feel pain in his or her entire mouth. In order to diagnose the problematic tooth, the dentist often must use tools and prodding techniques to induce even more pain at the site of the problematic tooth. Only then can the dentist know which tooth is at issue. Referring specifically now to FIG. 2, and as briefly noted above, a distraction apparatus 100′ can be configured as a mouthpiece to fit inside a user's mouth. Here, the mouthpiece 100′ can be configured both as a diagnostic tool, as well as a distraction tool.

The projections 106′ may be specifically located at points along the length of the mouthpiece generally corresponding to the locations of the patient's teeth. Rather than the dentist having to individually poke each tooth in the patient's mouth, the mouthpiece 100′ may be inserted by the patient, and the oscillation device 104′ may induce vibrations of the projections 106′. Each projection 106′ may be stimulated simultaneously. Preferably, the projections 106′ are stimulated individually (e.g., in succession), such that the dentist can precisely identify the problematic tooth. The stimulations may be activated by the dentist, such that there is a clear correlation between the oscillating projection 106′ and the tooth. Accordingly, the oscillation device 104′ may be in communication (e.g., wired or wireless) with a structure (e.g., structure 500, FIG. 10), which may be implemented for example on a computer or other mobile device comprising a program. Using the structure 500, the dentist may identify a projection 106′ to activate oscillation. The patient may or may not perceive the vibrations, although the vibrations must be substantial enough so as to allow the patient to alert the dentist to discomfort with a particular tooth.

In a further embodiment of the mouthpiece 100′, the projections 106′ may be non-specific to a particular location, and may be configured to oscillate in order to distract a patient from an impending treatment event. For example, many people experience more trauma from the shots given to numb an area of the mouth than from the dental work itself. Accordingly, the mouthpiece 100′ may be used to distract the patient from the impending injection, and may then be removed such that the doctor can complete the dental work.

In another embodiment, a preemptive pain avoidance apparatus 200 may be configured to pre-condition an individual's skin prior to a treatment event which may cause pain. In one example, the preemptive pain avoidance apparatus 200 resembles a bandage, such as a Band-Aid® Brand bandage, illustrated in FIGS. 5-9. Similar to known bandages currently available on the market, the bandage may include an adhesive area 202 and a pad 204.

The pad 204 may be pre-treated with one or more chemicals, such as a numbing agent (e.g., lidocaine, prilocaine, benzocaine, pramoxine, etc.). Because topical numbing agents often require several minutes to take effect, medical staffs are often apprehensive to use such agents for simple procedures, such as injections, even for children that have a difficult time understanding and adapting to pain experienced as a result of such injections. For some patients, the trauma experienced by receiving even simple procedures is significant, and the benefit of using such topical numbing agents is therefore unquantifiable.

To avoid the trauma, the pre-treated bandage 200 may be applied by an individual (or an individual's caretaker) to the individual's skin in the area in the area where the individual will receive the injection (or where another treatment event may occur). The bandage is applied such that the pad 204 comes into contact with the individual's skin. The individual may visit the doctor sometime after the bandage is applied, giving the chemical enough time to take effect without causing delays to the medical staff.

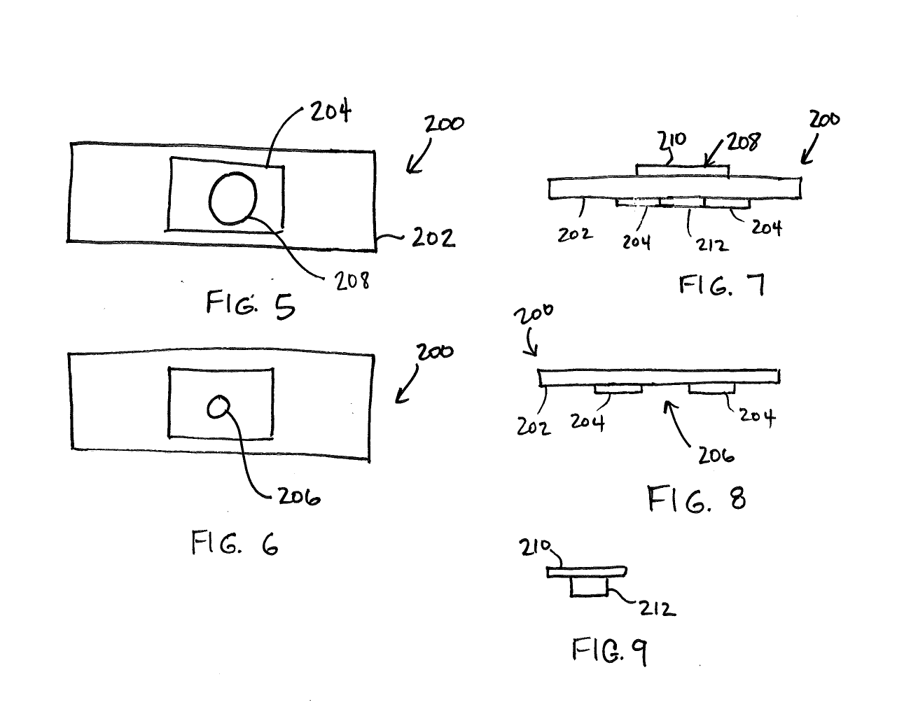

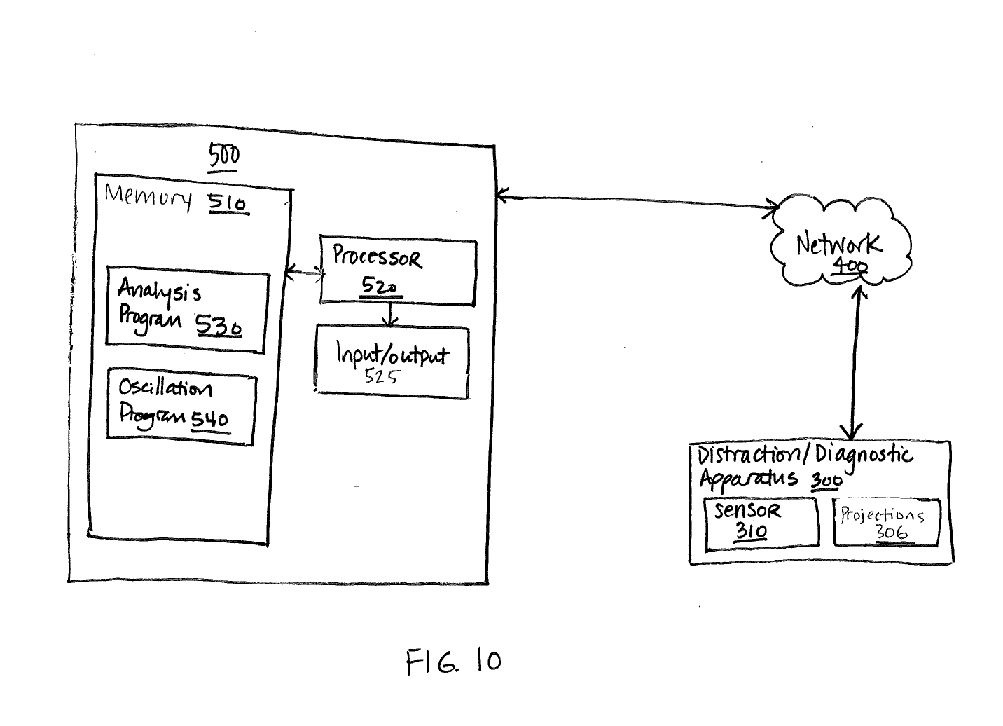

Optionally, the pad 204 may include a cutout 206 extending through the pad 204 and the top layer of the bandage 202, leaving a hole. A cap 208 may fit within the cutout and may further include an adhesive portion 210 defining a cap 208 and a plug 212. The cap 208 temporarily adheres to the bandage (FIG. 7), and the plug 212 may be composed of the same material as the pad 204, and have a numbing agent. The plug 212 extends through the opening 206 to provide the numbing agent to the individual at the area thereunder. The medical staff knows where precisely where pre-treatment of the skin occurred, as the cap 208 may be removed from the bandage 200, revealing the cutout 206 (FIGS. 6 and 8). The medical staff may then complete the injection within the cutout 206. This helps to ensure that the medical staff administers the injection at the appropriate location that has received pre-conditioning. In embodiments, the medical staff may provide instructions to the individual (or the individual's caretaker) for placing the bandage in the correct location for a particular treatment event (e.g., some injections are appropriately administered in the arm, while other injections are more appropriate in the glute).

In further embodiments, the bandage may be configured to provide electromagnetic, electrochemical, or electrical stimulus to the skin under the bandage 200. Electromagnetic therapy has previously been used in the treatment of neurological disorders. However, such therapy traditionally requires a visit to a practitioner specializing in such therapy. Here, the bandage may be equipped with electronics capable of generating a magnetic field in the area of the bandage. For example, the bandage 200 may include a coil (e.g., at or near the area of the pad). A stimulator, which may be energized via a battery, for example, may provide electric current to the coil. When the stimulator triggers the coil, a magnetic field may be generated near the skin, thereby stimulating the muscle.

It is known that electrical currents flow through nerves and muscles. Changes in the intensity or direction of the current causes muscles to contract. In an alternative embodiment, using devices and techniques known to those of skill in the art, the bandage may be equipped to provide low intensity electrochemical tremors to an individual's muscle in the area of the bandage 200. The low intensity, low voltage electrochemical tremors can be induced upon an individual's muscles to stimulate the muscle, causing the muscle to quiver. In embodiments, the quivering will be almost or entirely imperceptible to the individual. While the muscle is quivering, the medical professional may administer the injection. The individual may not feel the injection because the muscle is distracted by the stimulation. In still a further embodiment, the stimulation may be a simple low-voltage electrical stimulation that does not require the presence of a chemical or magnetic coil.

In such embodiments where the bandage 200 does not require application prior to visiting the medical professional, the bandage 200 may be applied by the medical professional. The electrical, electromagnetic or electrochemical reaction causing the stimulation may be triggered by an event, such as the removal of the backing from the bandage 200 or contact of the pad with a topical ointment applied prior to placement of the bandage. Once the bandage is in place, the electrochemical tremors may begin. The bandage here may also contain a cutout 206, as described above, to define the injection site for the medical professional to ensure that the injection occurs where preconditioning of the surface has occurred.

Of course, the bandage 200 may be configured based on the particular treatment event. A small injection may require only a small bandage and small muscular tremors to adequately distract the individual. For a more intense treatment event, the bandage 200 may be larger, or may be configured to provide a greater degree of tremors. It shall be understood that, in any event, the tremors are not so intense so as to cause an unintentional medical event. Furthermore, where such electrical, electrochemical, or electromagnetic reactions would be potentially averse to an individual (e.g., interact adversely with a medical device such as a pace maker), the user may be dissuaded from using the bandage.

FIG. 10 is a schematic illustration of a distraction and/or diagnostic apparatus 300 in communication with a structure 500. The apparatus 300 may be substantially similar to any of the apparatus 100, 100′, 100″, and/or 200 defined herein, and includes projections 306. Here, however, the apparatus 300 includes one or more sensors 310 which may be used to monitor the health of the individual. For example, electricity is often used to detect health problems, such as heart abnormalities, brain disturbances, or loss of nerve function. Accordingly, the bandage 200 equipped with a sensor may additionally be configured to receive information from the body. Other sensors that do not measure electrical impulses within the body may additionally, or alternately, be employed (e.g., temperature sensors, sweat sensors, etc.). According to another example, one or more sensors may be incorporated into the mouthpiece 100″ to measure bacteria levels in the mouth. Thus, various types of sensors 310 can be incorporated into the apparatus 300 to monitor information about the individual.

The sensor 310 may transmit (e.g., wirelessly over a network 400) the data to a structure 500, which may take the information from the body and provide relevant diagrams or other reporting means for a medical professional to review. The structure 500 includes non-transitory computer memory 510, a processor 520, an input/output device 525, and various programming as discussed below, including an analysis program 530 and an oscillation program 540. As noted above, the structure 500 may be configured as a computer or other mobile device having the components described herein.

The computer memory 510 may include volatile and non-volatile memory, and any appropriate data storage devices whether now existing or later developed may be used. Further, the computer memory 510 may be a unitary memory in one location, or may alternately be a distributed computer memory such that one portion of the computer memory is physically separate from another portion of the non-transitory computer memory. In other words, discrete computer memory devices may be linked together (e.g., over a network) and collectively form the computer memory 510. While this document often refers to elements in the singular, those skilled in the art will appreciate that multiple such elements may often be employed and that the use of multiple such elements which collectively perform as expressly or inherently disclosed is fully contemplated herein.

The processor 520 may be any appropriate device, whether now existing or later developed, which performs the operations specified by the various programming used by the structure 500. The processor 520 may be electronic circuitry located on a common chip or circuit board, or may be a distributed processor such that one portion of the processor is physically separate from another portion of the processor. The processor 520 is in data communication with the computer memory 510 and the input/output device 525.

The input/output device 525 may be any input and output device (whether now existing or later developed) allowing a person to input information for storage in the computer memory 510. For example, the input/output device 252 may be a touchscreen, a keyboard, a computer mouse, a microphone, or any other device which may be configured to receive information from a user and to transmit information back to the user. For example, the output 525 may provide visual/audible programming (either preset or selectable) and/or instructions (e.g., instructions on where to place a bandage 200). In embodiments, the input/output device 525 may be separate components.

The computer memory 510 has various programs to cause the sensor information to be analyzed (e.g., the analysis program 530), or to induce oscillations by the apparatus 300 (e.g., the oscillation program 540). More specifically, the analysis program 530 obtains information from the sensor(s) 310, which may be stored in the computer memory 510. The analysis program 530 may have a series of steps to analyze the sensor information, and provide an output in response thereto. The output may simply be a reading of the information taken by the sensor (e.g., amount and/or type of bacteria), which may be shown to the user for informational purposes. The output may further be a report, which may be sent (e.g., over the network 400) to a patient's primary care physician, or the patient's electronic medical record. Other outputs may be appropriate, and are considered within the scope of the invention.

The oscillation program 540 may include programming for automatically initiating oscillations (e.g., according to a pattern). Alternately, or additionally, the oscillation program 540 may include instructions for directing a user (e.g., a dentist) to identify a particular projection or projections 106 for oscillation in order to easily track the stimulation location.

From the foregoing, it shall be understood that the individual using the apparatus 300 may or may not be an active participant in the control thereof. The apparatus 300, which may form a dynamic control loop, may be managed locally (e.g., at the apparatus 300) or via distributed intelligence to a device 500 remotely located and in communication to the apparatus 300 (e.g., a cell phone, personal computer, etc.). Feedback of information to and from the apparatus 300 can be bi-directionally provided by sensor readings located at or near the apparatus, as well as by user inputs entered into the remote device 500, which may allow a user to modulate preemptive scaling of pain management or a level of numbness based on the pain experienced (or anticipated to be experienced) by the individual at the injection site, as well as stimulus signals provided by the apparatus 300. In other words, a user may place the apparatus 300 at a location on his or her body (e.g., arm), or at a location on another person's body, and, upon an indication of ready status, select a stimulus excitation mode to test the area for a user's individual perception of numbness. This iterative process could be saved to a user's preference profile (e.g., in the memory 510) in such a way that the system can determine whether the individual is ready to receive an injection. The individual may thus gain a level of confidence through the system, including with the injection process itself. Accordingly, the system may be influenced by both sensors 310 and an individual wearing or operating the apparatus 300 in order to create a biofeedback mechanism whereby pain management may be self-controlled.

Many different arrangements of the described invention are possible without departing from the spirit and scope of the claimed invention. Embodiments of the invention are described herein with the intent to be illustrative rather than restrictive. Alternative embodiments will become apparent to those skilled in the art that do not depart from its scope. A skilled artisan may develop alternative means of implementing the disclosed improvements without departing from the scope of the invention.

Further, it will be understood that certain features and subcombinations are of utility and may be employed without reference to other features and subcombinations and are contemplated within the scope of the claims. Not all steps listed in the various figures and description need to be carried out in the specific order described. The description should not be restricted to the specific described embodiments.

1. A method for distracting an individual from a treatment event, comprising:
providing a distraction tool, comprising:
a stimulation apparatus comprising a housing and a plurality of projections extending from the housing; and
an oscillation device in communication with the stimulation apparatus;
positioning the distraction tool at a contact location on an individual, the contact location being substantially adjacent a location of a treatment event;
activating the oscillation device to induce vibrations in the stimulation apparatus; and
subsequently initiating the treatment event at the treatment event location;
wherein activating the oscillation device to induce vibrations causes the plurality of projections to oscillate against the individual at the contact location, thereby shifting attention from the treatment event to the vibrations, wherein the shift in attention causes the individual to discount pain caused by the treatment event.

2. The method of claim 1, wherein the projections are rigid.

3. The method of claim 2, wherein the stimulation apparatus is configured as a mouthpiece.

4. The method of claim 3, wherein each projection is disposed at a location of the mouthpiece generally corresponding to a location of a tooth of the individual.

5. The method of claim 1, wherein the stimulation is configured as a band to wrap around an appendage of the individual.

6. The method of claim 1, wherein the housing comprises a cutout, and wherein the treatment event location is situated within the cutout.

7. The method of claim 6, wherein the treatment event is an injection.

8. The method of claim 1, wherein the distraction tool further comprises a sensor.

9. The method of claim 8, wherein the sensor is in data communication with a structure comprising computer memory comprising programming for analysis sensor information from the sensor.

10. The method of claim 1, wherein the projections are disposed on opposing top and bottom sides of the housing.

11. The method of claim 10, wherein the projections are at least one of rigid and flexible.

12. A method for distracting an individual from a treatment event, comprising:
providing a distraction tool, comprising:
a stimulation apparatus; and
an oscillation device in communication with the stimulation apparatus;
positioning the distraction tool at a contact location on an individual;
activating the oscillation device to induce vibrations in the stimulation apparatus; and
commencing a treatment event to the individual;
wherein activating the oscillation device to induce vibrations causes the distraction tool to vibrate against the individual, thereby shifting attention from the treatment event to the vibrations, wherein the shift in attention causes the individual to discount pain caused by the treatment event.

13. The method of claim 12, wherein the oscillation device is integral with the stimulation apparatus.

14. The method of claim 13, wherein the oscillation device is in data communication with a structure comprising programming that, when effectuated by a processor, performs steps for causing the oscillation device to vibrate according to a predetermined pattern.

15. The method of claim 13, wherein the oscillation device is in data communication with a structure comprising an input device for receiving instructions from a user and programming that, when effectuated by a processor, performs steps for causing the oscillation device to vibrate according to the instructions from the input device.

16. The method of claim 12, wherein the stimulation apparatus further comprises a sensor.

17. The method of claim 16, wherein the stimulation apparatus is in data communication with a structure comprising programming that, when effectuated by the processor, performs steps for retrieving data from the sensor and analyzing the data from the sensor to determine an output data.

18. The method of claim 17, wherein the structure further comprises an output device, and wherein the output device displays the output data.

19. The method of claim 12, wherein the stimulation apparatus further comprises at least one of a heating and cooling element.

20. A system for diagnosing and providing a distraction to an individual, comprising:
a distraction tool, comprising:
a stimulation apparatus comprising a housing and a contact area having a distraction mechanism; and
a sensor; and
a structure comprising an input/output device and a processor in data communication with non-transitory computer memory comprising a program that, when effectuated by the processor, performs the following steps:
receive a first input from the input/output device to determine a first level of distraction;
activate the distraction mechanism of the stimulation apparatus based on the first level of distraction;
receive a second input from the input/output device to determine a second level of distraction;
adjust the distraction mechanism of the stimulation apparatus based on the second level of distraction;
receive a sensor input from the sensor;
analyze the sensor input based on a predetermined set of conditions; and
provide an output at the input/output device, the output being based on the sensor input analysis.
